(19) United States
(12) Patent Application Publication
(10) Pub. No.: US 2014/0142572 A1
(43) Pub. Date: May 22, 2014

(54) Water Evacuating Tube for Washing Electric Knife

(71) Applicant: Fuji Systems Corporation, Tokyo (JP)

(72) Inventors: Yoshihiko Maehara, Fukuoka (JP); Tetsuo Ikeda, Fukuoka (JP); Junichi Ugawa, Kanagawa (JP)

(73) Assignee: Fuji Systems Corporation, Tokyo (JP)

(21) Appl. No.: 14/084,790

(22) Filed: Nov. 20, 2013

(30) Foreign Application Priority Data
Nov. 21, 2012 (JP) 2012-254913
Feb. 5, 2013 (JP) 2013-020065

(51) Int. Cl. A61B 18/14

(52) U.S. Cl. CPC A61B 18/1482; A61B 18/1402
USPC 606/46; 606/45

(57) ABSTRACT

A water evacuating tube for washing an electric knife of the present invention includes a heat resistant tube main body, in which front and rear ends are open, and an inside in an axial direction is a hollow portion in which the electric knife is disposed and held. In a peripheral wall of the tube main body, a lumen for pouring water is disposed in the axial direction, and a front end portion of the tube main body is provided with a pouring water aperture for dropping the water from the lumen onto an electrode of the electric knife.

### BACKGROUND OF THE INVENTION

The present invention relates to a water evacuating tube for washing an electric knife.

In an endoscopic operation as well as a general operation, an electric knife called monopolar or bipolar is used for the resection and stopping of bleeding of the affected part. This electric knife applies a high-frequency current between two electrodes to generate a Joule heat by the contact resistance, thereby causing a cell to vaporize to make an incision, or evaporating water in a cell and coagulating a protein to stop the bleeding. In order to stably use the function of the electric knife during an operation, it is necessary to remove an adhesive protein which has adhered to the electrode and denatured by heat.

However, in such an endoscopic operation, in order that the protein may be removed which has adhered to the electrode of the electric knife, once the electric knife must be brought from a trocar and the like to the outside of the body, the electrode must be washed and then reinserted into the body, with the result that the work is complicated. In a hepatectomy in particular, because the liver is a vascular-abundant parenchymatous organ, the bleeding stopping effect and coagulating effect are very important. For example, when the electric knife is even briefly brought to the outside of the body, the bleeding from the liver parenchyma may deteriorate the surgical field, as well as the blood may reduce the coagulating effect.

In view of such a situation as described above, the inventors started the development of a technology to remove a protein which had adhered to an electrode without bringing the electric knife to the outside of the body, and have completed the present invention. Examples of a patent document considered to serve as reference in relation to the present invention include JP 2008-093173 A (Patent Document 1) and JP 2011-156383 A (Patent Document 2).

Patent Document 1 as described above relates to an endoscope cleaning sheath and an endoscope having the same, which spew a gas-liquid mixing fluid of air and water fed from a liquid supply hole to the viscous liquid, blood, fat and the like which are derived from the living body and have adhered to the top end surface of an insert portion during an autoscopic observation, thereby removing them. According to Patent Document 1, the object to be washed is only the top end surface of the insert portion of the endoscope washing sheath. Furthermore, a lumen for feeding both air and water must be mounted on a tube body, a tube for feeding the air and a tube for feeding the water into the lumen are individually required, and the like, with the result that the structure is complicated. Patent Document 2 also relates to a fluid-assisted electrosurgical device, an electrosurgical unit with a pump and a method of use thereof, including a handheld electrosurgical device. In Patent Document 2, to electrodes of this device, saline which is a fluid from a fluid outlet is fed to prevent a bio tissue from adhering to the surface of the electrodes. According to Patent Document 2, lumens or tubes for feeding water to the respective electrodes are required, and each of the electrodes themselves to which the fluid is fed requires a construction suitable for receiving the fluid, such as a fluid outlet and recess portion.

### SUMMARY OF THE INVENTION

The objective of the present invention is, in view of the conventional problems described above, to provide a water evacuating tube for washing an electric knife, which is, without the need for the adoption of a conventional complicated structure or construction, directly applicable to a conventional electric knife because of its simple structure. Furthermore, in the water evacuating tube of the present invention, the electric knife is not brought to the outside of the body to be washed, but water is continuously dropped onto the electrode to wash the electrode, thereby preventing an adhesive protein from adhering to the electrode as well as cooling the electrode.

In order to achieve the above objective, a water evacuating tube for washing an electric knife of the present invention is used to feed water to wash or cool the electric knife, including a heat resistant tube main body, in which front and rear ends are open, an inside in an axial direction is a hollow portion in which the electric knife is inserted and held, in a peripheral wall a lumen for pouring water is disposed in the axial direction, and a front end portion is provided with a pouring water aperture for dropping the water from the lumen onto an electrode of the electric knife.

The present invention having the above construction allows, without bringing the electric knife to the outside of the body to wash the electrode each time like a conventional manner, dropping the water continuously from the pouring water aperture onto the electrode to wash the electrode, thereby preventing an adhesive protein from adhering to the electrode. In addition, the electrode may be cooled with the water from the pouring water aperture in parallel. Furthermore, because the present invention only includes the tube main body, in the peripheral wall of which the lumen for pouring water is disposed, and a front end portion of which is provided with a beak piece having the pouring water aperture at a top end thereof, the present invention exhibits an excellent effect that the structure is simple and directly applicable to an existing conventional electric knife.

### BRIEF DESCRIPTION OF THE DRAWINGS

FIG. 1 is a partly omitted elevation view of the water evacuating tube for washing an electric knife of an embodiment of the present invention;

FIG. 2 is a plan view;

FIG. 3 is a bottom view;

FIG. 4 is a longitudinal sectional elevation view;

FIG. 5 is an enlarged sectional view of the top end;

FIG. 6 is an enlarged sectional view taken along VI-VI line in FIG. 5;

FIG. 7 is an enlarged sectional view taken along VI-VI line in FIG. 5, illustrating an alternate embodiment of the closure strip;

FIG. 8A is an elevation view illustrating the top end side of the water evacuating tube equipped with an electric knife;

FIG. 8B is a plan view illustrating the top end side of the water evacuating tube equipped with an electric knife;

FIG. 8C is an elevation view illustrating the rear end side of the water evacuating tube equipped with an electric knife;

FIG. 9 is an elevation view illustrating the rear end side of an alternate embodiment of the water evacuating tube equipped with an electric knife;

FIG. 10 is an enlarged sectional view of the branching connector portion;

FIG. 11 is a right side view of FIG. 10; and

FIG. 12 is a view illustrating the action in a usage state where the water evacuating tube equipped with an electric knife is inserted into a trocar.

### DETAILED DESCRIPTION OF THE INVENTION

Embodiments of the water evacuating tube for washing an electric knife of the present invention are described below referring to the drawings.

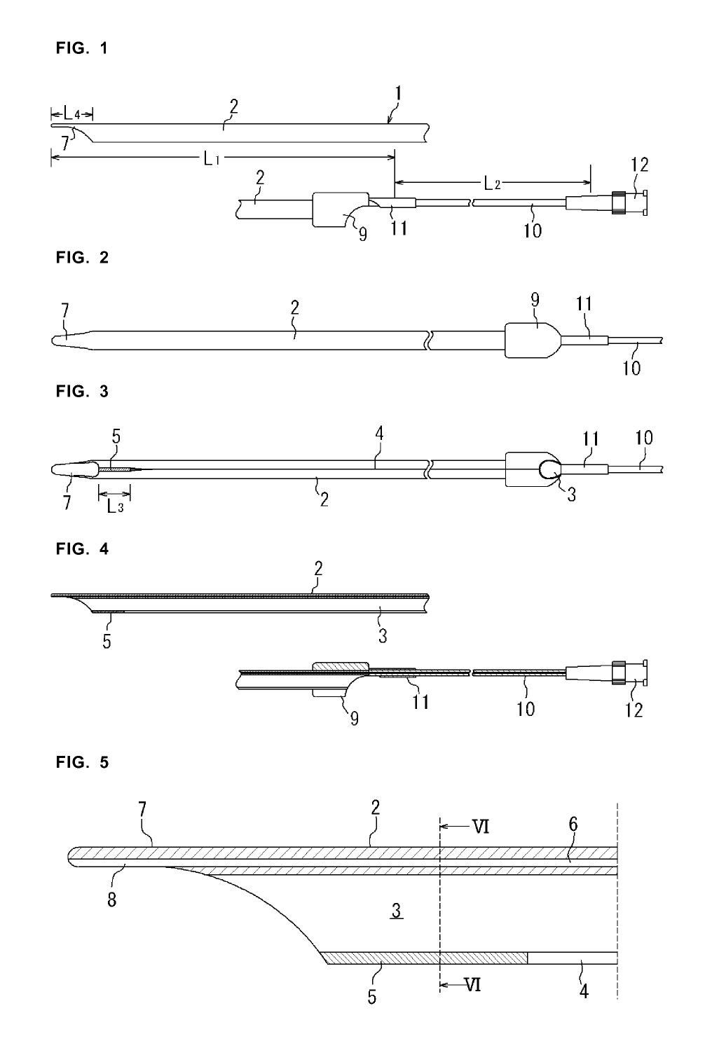

In FIGS. 1 to 4, reference number 1 represents a water evacuating tube which is used to wash an electric knife. The water evacuating tube 1 is used to feed water to wash or cool the electric knife in an endoscopic operation, is made of a flexible heat resistant resin such as silicone gum, and includes a tube main body 2 of a predetermined length with the front and rear ends open. The inside of the tube main body 2 in the axial direction is formed as a hollow portion 3 in which the electric knife is inserted and held. The wall thickness of the tube main body 2 is so thin that the tube main body 2 maybe inserted into a trocar and the like while being equipped with the electric knife as described below. In addition, because the tube main body 2 is made of the flexible heat resistant resin as described above, even when the tube main body 2 comes into contact with an electrode by some chance, the damage of the tube main body 2 may be prevented, and when the front end portion comes into contact with a bio tissue, the damage of the tissue may be prevented.

In the peripheral wall of the tube main body 2 which is located below in FIG. 1, also as illustrated in FIGS. 3 to 5, an axial slit 4 is formed almost over the entire length. The opening degree of the hollow portion 3 may be adjusted along the slit 4. In other words, the hollow portion 3 may receive the electric knife even in a case where the outer diameter of the electric knife is slightly larger than the inner diameter of the hollow portion 3. In FIGS. 5 and 6, reference number 5 represents a closure strip made of an elastic material such as silicone gum for closing a part of the slit 4. The closure strip 5 is mounted along the slit 4 on the line of the slit 4 so as to form the peripheral wall of the front end portion of the tube main body 2 in which the slit 4 is formed. In this way, the tube main body 2 has a structure configured not to open the slit 4 around the closure strip 5, thereby expanding or contracting so as to receive the electric knife to be inserted and then fixedly holding the received electric knife so as to prevent the holding from being impossible. Furthermore, as can be seen in FIGS. 3 and 6, because a part of the closure strip 5 to hold the slit 4 is embedded into a partially cut part of the slit 4 such that the thickness of the closure strip 5 is equal to the peripheral wall thickness, not only the inner peripheral surface but also the outer peripheral surface have no irregularity, with the result that the insertion into a trocar is smooth and the insertion performance is improved.

The peripheral wall of the tube main body 2 at the opposite side of the slit 4 is slightly thicker, in which a lumen 6 for pouring water is formed in the axial direction over the entire length. The lumen 6 for pouring water has, as illustrated in figures, an elliptical shape in cross-section extending in the circumferential direction so that the peripheral wall does not become too much large. However, this elliptical shape is just one example, and alternatively the lumen 6 may also have a circular arc in cross-section, be composed of a mass of small circular lumens arranged in parallel, or be more flat in cross-section.

FIG. 7 illustrates an alternate embodiment of the closure strip 5 as described above. In this embodiment, the slit 4 is formed so as to reach the front end opening of the tube main body 2, and onto the inner peripheral surface of the front end portion of the tube main body 2 in which the slit 4 is formed, a closure strip 5′ made of an elastic material is attached at the same position as that of the closure strip 5. The closure strip 5′ has the same function as that of the closure strip 5. In this case, even after the tube main body 2 with the slit 4 is produced, the closure strip 5′ may be mounted. Accordingly, the mounting of the closure strip 5′ becomes easier than that of the closure strip 5.

The closure strip 5 is mounted such that the thickness thereof is equal to that of the peripheral wall of the tube main body 2, while the closure strip 5′ is mounted on the inner peripheral surface of the tube main body 2. In either case, because the closure strip 5, 5′ is not mounted on the outer peripheral surface of the tube main body 2, there is no resistance when the water evacuating tube 1 equipped with the electric knife is inserted into a trocar and the like. Therefore, the insertion performance of the water evacuating tube 1 into a trocar and the like is enhanced.

The front end portion of the tube main body 2 in which the lumen 6 for pouring water is formed in the longitudinal direction of the peripheral wall as described above is integrally provided with a beak piece 7 which has the narrower top end to become a beak shape. The top end of the beak piece 7 is provided with an opened pouring water aperture 8 that is in communication with the lumen 6 for pouring water to spew and pour water from the lumen 6. Because the front end portion of the tube main body 2 is the beak piece 7, that is in the shape like the tip of a fountain pen, when the electric knife is mounted as described below, the poured water may be easily dropped onto electrodes as blades that are openable/closable to right and left and positioned just beneath the front end portion of the tube main body 2 to wash or cool the electrodes, and the opening and closing of the electrodes are not interrupted. In addition, the front end of the tube main body 2 is strengthened so that the shape of the tube main body 2 may be easily maintained, and the top end of the tube main body 2 may be prevented from bending down to be caught between the blade electrodes of the electric knife.

As can be seen in FIG. 4, the rear end portion of the tube main body 2 on the opposite side to the side of the beak piece 7 is provided with a beak member 9 which has the same beak shape as that of the beak piece 7. Also in the peripheral wall which is located below the beak member 9, an axial slit is formed over the entire length so as to overlap with the axial slit 4. At the top end (the right end in FIG. 4) of the beak member 9, one end portion of a tube 10 for pouring water is connected to the lumen 6 for pouring water in communication with each other. The part 11 of connection is strengthened with a double tube construction so as to prevent the rear end opening portion of the tube main body 2 from rolling up when the electric knife is inserted, and so as not to weaken the connection . In addition, the other end portion of the tube 10 for pouring water is provided with a closer 12 that is connected to a bag for pouring water (not illustrated) . Accordingly, water poured from the bag for pouring water goes through the tube 10 for pouring water into the lumen 6 for pouring water of the tube main body 2, and the water further passes through the lumen 6 for pouring water and is then poured out of the pouring water aperture 8 at the top end.

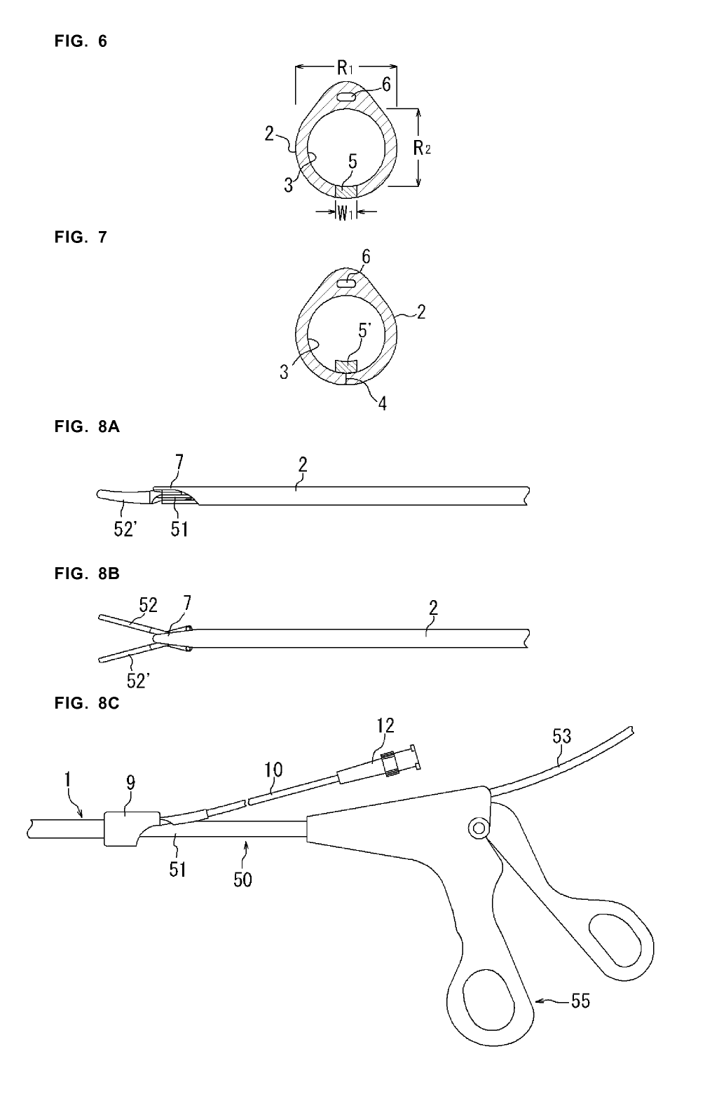

In FIGS. 8A, 8B and 8C, reference number 50 represents a conventionally well-known electric knife in a state of being mounted in and covered with the water evacuating tube 1. Briefly, the electric knife 50 is configured to have a rod body 51 made of metal in a predetermined length at least longer than the length of the tube main body 2, the top end portion thereof being formed as bifurcated blade electrodes 52, 52′ . The placement relationship between the beak piece 7 of the tube main body 2 and the blade electrodes 52, 52′ is such that the base end portion of the blade electrodes 52, 52′ is positioned just beneath the top end of the beak piece 7, and the blade electrodes 52, 52′ openable/closable to right and left faces ahead of the top end of the beak piece 7 so that water is easily poured. In other words, water from the pouring water aperture 8 at the top end of the beak piece 7 may be dropped onto the blade electrodes 52, 52′ every time the blade electrodes 52, 52′ are opened or closed. In addition, an electric wire 53 is wired along the rod body 51 so that a high-frequency current may be passed through the blade electrodes 52, 52′ during an operation.

When the water evacuating tube 1 is equipped with the electric knife 50, a part of the electric knife 50 projecting from the rear end opening portion of the tube main body 2 is provided with a scissors type operating handle 55. Grasping and opening/closing the handle 55 allow the blade electrodes 52, 52′ to open/close. As to the handling and the opening/closing of the blade electrodes 52, 52′, for example, opening the handle 55 pulls an inner rod (not illustrated) that is slidably disposed in the rod body 51 thereby closing the blade electrodes 52, 52′, and closing the handle 55 pushes the inner rod thereby opening the blade electrodes 52, 52′. However, this is only an exemplary opening/closing mechanism of the blade electrodes 52, 52′by the handle 55, and another opening/closing mechanism may be used.

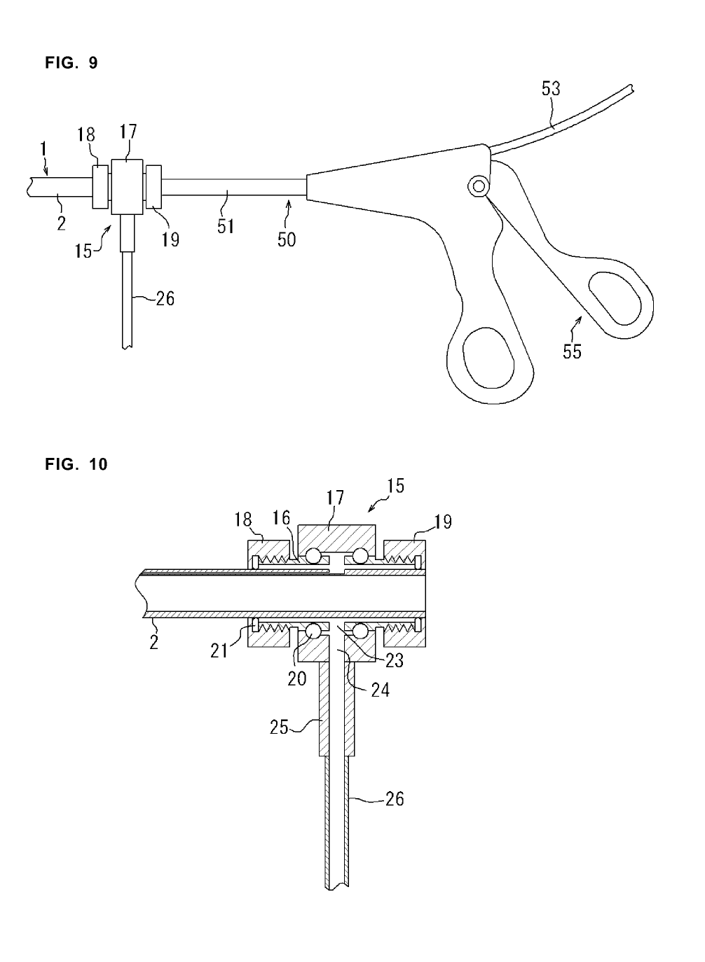

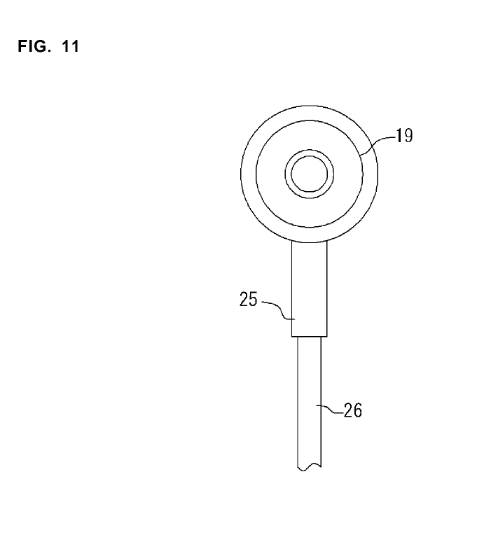

FIGS. 9 to 11 represent an alternate embodiment of the water evacuating tube 1. In the embodiment, the rear end portion of the tube main body 2 on the opposite side to the side of the beak piece 7 is equipped with, instead of the beak member 9 and tube 10 for pouring water, a branching connector 15 rotatably around the tube main body 2. The branching connector 15 is composed of a tubular main body 16 which is inserted into the rear end portion of the tube main body 2, a rotatable tubular body 17 which is inserted into the tubular main body 16 rotatably, and tubular clamping screws 18, 19 which are fixed by the thread engagement to both sides of the rotatable tubular body 17 and to both end portions of the tubular main body 16. Reference numbers 20, 21 represent a sealing O ring and a gasket. Through the peripheral wall of the tubular main body 16, a plurality of side holes 23 are provided in the circumferential direction. In addition, through the peripheral wall of the rotatable tubular body 17, a hole 24 communicating with one of the side holes 23 is provided. Furthermore, from the peripheral wall of the rotatable tubular body 17, a connecting pipe 25 is projected to communicate with the hole 24. To the connecting pipe 25, one end portion of a tube 26 for pouring water is connected in communication with each other through the lumen 6 for pouring water of the tube main body 2, the hole 24 and the one of the side holes 23. Furthermore, the other end portion of the tube 26 for pouring water is provided with a closer that is the same as the closer 12 connected to the tube 10 for pouring water as described above. According to the construction as described above, in the water evacuating tube 1 of this embodiment, water poured from the bag for pouring water goes through the tube 26 for pouring water, the connecting pipe 25, the hole 24 and the side hole 23 into the lumen 6 for pouring water of the tube main body 2, and the water further passes through the lumen 6 for pouring water and is then poured out of the pouring water aperture 8 at the top end. When pouring the water, the water tight is kept by the O ring 21 and the gasket 22.

Because using the rotatable branching connector 15 as described above may bend the tube 26 for pouring water in any direction, the tube 26 for pouring water that is given to tangling around the tube main body 2 thereby becoming a hindrance during an operation may be efficiently directed in a direction so as not to hinder the operation, with the result that the usability is improved.

The dimension of each portion of the water evacuating tube 1 in this embodiment may be indicated for reference as follows: a full length L₁ of the tube main body 2 is around 300 mm, a full length L₂ of tube 10 for pouring water is around 200 mm, a length L₃ of the closure strip 5 in the axial direction is around 10 mm, a width W₁ thereof is around 1 mm, a length L₄ of the beak piece 7 in the axial direction is around 15 mm. In addition, an outer diameter R₁ of the tube main body 2 is around Φ5.8 and an inside diameter R₂ thereof is around Φ5.0. Furthermore, a diameter of the lumen 6 for pouring water is around 0.3 mm at the small diameter side and around 1 mm at the large diameter side (see FIGS. 1, 3 and 6).

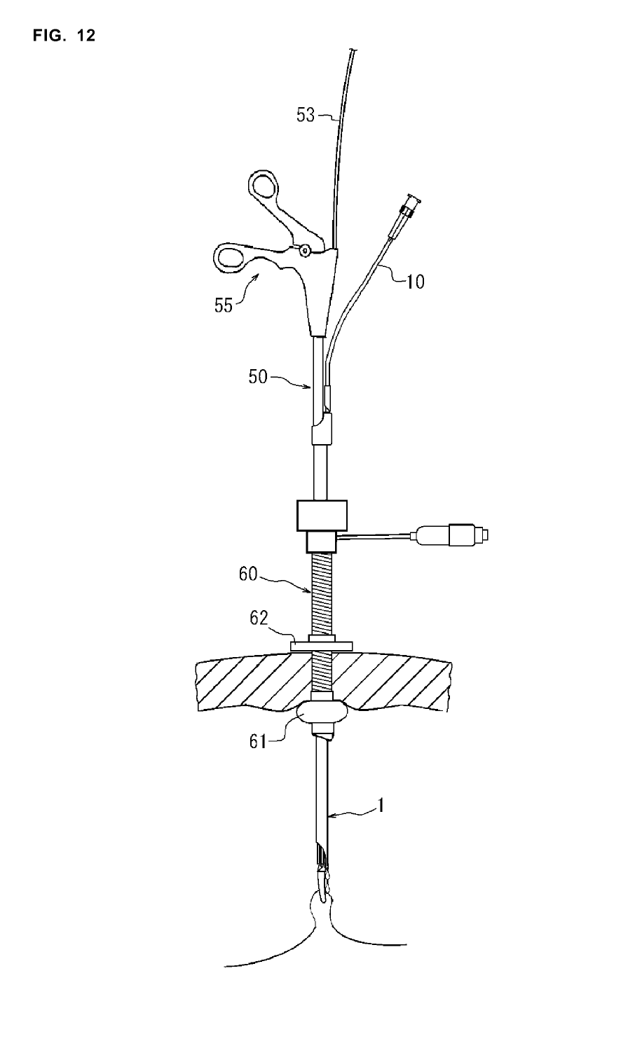

Next, a description is made of a method of use for the water evacuating tube 1 with reference to FIGS. 8A to 8C and 12.

When the water evacuating tube 1 is used, first of all, the electric knife 50 is inserted from the side of the top end thereof at which the blade electrodes 52, 52′ are disposed into the hollow portion 3 of the tube main body 2 starting from the side of the rear end opening portion thereof. As to the insertion, because the inside diameter of the hollow portion 3 is generally configured to match with the outer diameter of the rod body 51 of the electric knife 50, the electric knife 50 can be inserted without the slit 4 open. Herein, even when the outer diameter of the rod body 51 of the electric knife 50 to be inserted is slightly larger than the inside diameter of the hollow portion 3, the slit 4 is opened so that the hollow portion 3 is slightly expanded to allow the insertion. When the outer diameter of the rod body 51 of the electric knife 50 is slightly larger than the inside diameter of the hollow portion 3, the insertion is performed in such a way that the closure strip 5 is extended. Then, as illustrated in FIGS. 8A and 8B, when the blade electrodes 52, 52′ appear on the outside of the beak piece 7, the insertion is stopped and the positioning is performed. By this positioning, the electric knife 50 is held and mounted in the water evacuating tube 1. At this time, the closure strip 5 which has been extended by the insertion shrinks back to its original state, thereby firmly holding the rod body 51 of the electric knife 50 at that position. The position of the operating handle 55 and the like of the electric knife 50 when being mounted in the water evacuating tube 1 is in a state as illustrated in FIG. 8C.

After the electric knife 50 is mounted in the water evacuating tube 1 as described above, the abdomen is punctured with a trocar 60 that is inserted to a predetermined position and fixed there with a balloon 61 and stopper 62, as illustrated in FIG. 12. Into the trocar 60 mounted in this manner, the water evacuating tube 1 equipped with the electric knife 50 as described above is inserted from the top end side thereof. This is in a state before an operation, from which the operator grasps the operating handle 55 of the electric knife 50 and performs the operation while observing an affected part in the bio tissue using an endoscope (not illustrated) . FIG. 12 illustrates a situation in which an affected part of the bio tissue is picked, cut and the like with the blade electrodes 52, 52′ of the electric knife 50 during an operation.

In the water evacuating tube 1, during the operation, water derived from the lumen 6 for pouring water of the tube main body 2 is always poured so as to spew from the pouring water aperture 8 of the beak piece 7, and the water is dropped onto the blade electrodes 52, 52′ of the electric knife 50 positioned just beneath the pouring water aperture 8. Accordingly, conventionally there was a problem in that the blade electrodes 52, 52′ were suffered from the adhesion of an adhesive protein, however, the poured water washes away the adhesive protein so as to prevent the adhesion, with the result that the electrodes 52, 52′ may be always maintained in a cleanly washed state. In addition, although the blade electrodes 52, 52′ through which a high-frequency current passes are at high temperatures, they are cooled with the poured water in parallel. Therefore, also in view of the aspect that the tube main body 2 is made of a heat resistant resin, the top end of the tube main body 2 is prevented from bending down to be caught between the blade electrodes 52, 52′ of the electric knife 50, with the result that a satisfactory usage state may be maintained over a long period of time.

As described above, using the water evacuating tube 1, without bringing the electric knife 50 to the outside of the body to wash the blade electrodes 52, 52′ each time like a conventional manner, may drop water continuously from the pouring water aperture 8 of the beak piece 7 onto the blade electrodes 52, 52′ to wash the blade electrodes 52, 52′, thereby preventing an adhesive protein from adhering to the electrodes 52, 52′. In addition, the blade electrodes 52, 52′ may be cooled with the water from the pouring water aperture 8 in parallel. In addition, because the axial slit 4 is formed almost over the entire length so as to connect the front and rear ends of the tube main body 2, the tube main body 2 may receive and cover the electric knife 50 even with the slightly larger outer diameter. In addition, because the elastic closure strip 5, 5′ is mounted, even when the tube main body 2 receives the electric knife 50 with the slightly larger outer diameter, the tube main body 2 may effectively hold the electric knife 50 without spreading the slit 4 at the front end portion of the tube main body 2. In addition, because the tube 10 for pouring water is mounted in communication with the lumen 6 for pouring water, using the tube 10 may continuously feed water. In addition, with the use of the water evacuating tube 1 as illustrated in FIGS. 9 to 11, because the branching connector 15 is rotatably mounted around the tube main body 2, and on the connector 15, the tube 26 for pouring water is mounted in communication with the lumen 6 for pouring water of the tube main body 2, the tube 26 for pouring water no longer tangle around the tube main body 2 during an operation, with the result that the operation may be smoothly performed.

Note that, in the above embodiment, the water evacuating tube 1 is described in which the slit 4 is formed over the entire length of the tube main body 2, however, the slits 4 is not necessarily formed. This is because, in a case where the inside diameter of the hollow portion 3 of the tube main body 2 is equal to or slightly larger than the outer diameter of the electric knife 50, the insertion is easily performed. Each of the closure strips 5, 5′ is only shown as a preferable example, and any other embodiments, including a ring-shaped one in the circumferential direction for example, may be used as long as the same function and the same effect are achieved. Also, the water evacuating tube 1 shown as an embodiment is only a preferable example. It goes without saying that the detail of design and the like of the present invention may be arbitrarily changed or modified within the scope of the claims.

1. A water evacuating tube for washing an electric knife used to feed water to wash or cool the electric knife, comprising
a heat resistant tube main body, in which front and rear ends are open, an inside in an axial direction is a hollow portion in which the electric knife is disposed and held, in a peripheral wall a lumen for pouring water is disposed in the axial direction, and a front end portion is provided with a pouring water aperture for dropping the water from the lumen onto an electrode of the electric knife.

2. The water evacuating tube according to claim 1, wherein a front end portion of the tube main body is provided with a beak piece, and a top end of the beak piece is provided with the pouring water aperture.

3. The water evacuating tube according to claim 2, wherein the peripheral wall of the tube main body is provided with an axial slit almost over an entire length such that front end opening and rear end opening of the tube main body are connected to each other.

4. The water evacuating tube according to claim 3, wherein the peripheral wall of the tube main body near the beak piece is provided with an elastic closure strip for closing the axial slit.

5. The water evacuating tube according to claim 2, wherein a rear end portion of the tube main body on an opposite side to the beak piece is provided with a tube for pouring water so as to communicate with the lumen for pouring water and pour the water into the lumen.

6. The water evacuating tube according to claim 2, wherein a rear end portion of the tube main body on an opposite side to the beak piece is provided with a branching connector rotatably around the tube main body, and wherein to the branching connector, a tube for pouring water is attached so as to communicate with the lumen for pouring water of the tube main body.

7. The water evacuating tube according to claim 6, wherein the branching connector has a tubular main body in which the tube main body is disposed, a rotatable tubular body in which the tubular main body is rotatably disposed, and tubular clamping screws which are fixed by thread engagement to both sides of the rotatable tubular body and to both end portions of the tubular main body, and wherein through a peripheral wall of the tubular main body, a plurality of side holes are provided in a circumferential direction, through a peripheral wall of the rotatable tubular body, a hole configured to communicate with one of the side holes is provided, and to the hole, one end portion of the tube for pouring water is attached in communication through the lumen for pouring water of the tube main body, the hole and the one of the side holes.

8. The water evacuating tube according to claim 1, wherein the electric knife is used in an endoscopic operation.

9. The water evacuating tube according to claim 2, wherein the electric knife is used in an endoscopic operation.
